(19) United States
(12) Patent Application Publication
(10) Pub. No.: US 2026/0002902 A1
(43) Pub. Date: Jan. 1, 2026

(54) BIOSENSOR APPARATUS

(71) Applicants: EPINAL LIMITED, Reading, Berkshire (GB); UNIVERSITY OF MANCHESTER, Manchester (GB)

(72) Inventors: Michael L. Turner, Manchester (GB); John Rudin, Bristol (GB)

(21) Appl. No.: 18/868,639

(22) PCT Filed: Apr. 24, 2024
(86) PCT No.: PCT/EP2024/061252

§ 371 Date: Nov. 22, 2024

(30) Foreign Application Priority Data
May 17, 2023 (GB) 2307350.5

(51) Int. Cl. G01N 27/327; G01N 27/414; G01N 33/543

(52) U.S. Cl. CPC G01N 27/3276; G01N 27/4145; G01N 33/5438

(57) ABSTRACT

A structure comprises multiple individually removable functionalised electrodes, each one of the multiple individually removable functionalised electrodes for use in at least one biosensor apparatus.

### TECHNICAL FIELD

The disclosure relates generally to the field of biosensors, gas sensors, and chemical sensors where multiple different sensitive coatings are required in the same device. Aspects of the disclosure relate to manufacturing functionalised electrodes for use in a biosensor.

### BACKGROUND

FIG. 1 shows an Odorant Binding Protein (OBP) 100, for understanding of the invention. Biosensing using OBPs has been developed during the past 20 years. OBPs are a class of proteins found in the olfactory systems of vertebrates and insects. Due to their folding arrangement, OBPs have high temporary binding affinities to specific chemicals. These can be genetically modified from naturally occurring proteins, or be fully synthesised, to augment their binding specificity. When binding occurs, the electrical characteristics (for example, charge and dielectric properties) change, which then can be amplified and used as a sensor input.

One means of providing sensitive amplification is to incorporate the OBP as a layer on the gate electrode of an Electrolyte Gated Field Effect Transistor (EGFET), particularly those which use organic semiconductors (EGOFET). Other modes such as simple electrolytic capacitance measurements and optical effect may also be used.

FIG. 2 shows a typical EGOFET arrangement for understanding of the invention. The EGOFET 200 comprises a source 201 and drain 202 electrodes with a semiconductor layer 203 forming the channel of the FET, and a separate gate electrode 204. The charge on the gate electrode 204 is transferred via the electrolyte 205 to the semiconductor surface, inducing charge carriers allowing a current flow in the device. A layer of active sensing material 206 is immobilised on the gate electrode 204 and analytes contained in the electrolyte 205 may bind directly to the sensing material and modify the field applied to the semiconductor channel. By biasing the voltage on the gate 204 and source/drain electrodes, a significant amplification of the electrical change induced by binding can be afforded, and an extremely sensitive sensor produced. The analyte/electrolyte 205 flows to the device by arranging the substrates 210, 211 to form microfluidic channels 212. Sensing materials may comprise, but are not limited to, OBPs, DNA, Antibodies, proteins or enzymes. The electrolyte/analyte fluid is typically presented to the EGOFET devices using microfluidics, and one advantage of these devices is that they can be fabricated on a small (50-500 um) length scale.

A typical application for such a sensitive specific sensor is in narcotics detection, e.g. detecting at nanomolar (nM) or picomolar (pM) concentrations tetrahydrocannabinol (THC) in the presence of many interfering compounds, and discriminating it from cannabidiol (CBD), which has the same chemical composition, but a different and non-psychoactive structure.

Whilst the sensing devices have very high sensitivity, in order to discriminate and detect multiple materials, a range of biosensing materials must be used within the same sensor and exposed to the same analyte sample. Coating many different biological materials into the same microfluidic system presents a number of significant challenges. For example, a typical narcotics sensor based on OBPs might require 5-10 different proteins to be immobilised on 50-100 devices, allowing for redundancy, concentration ladders and calibration/reference devices. Immobilisation of the bioactive material typically consists of a number of steps carried out in solution allowing significant time (hours) for immobilisation and high surface coverage to occur. In the prior art, only small numbers of different sensing materials have been directly applied to gate electrodes in parallel on the same substrate, risking cross contamination and requiring wet processing of the whole substrate.

### SUMMARY OF THE INVENTION

An objective of the present disclosure is to provide means to overcome the production and customisation of sensor arrays by processing the gate electrodes separately, using wafer or tape scale processes, and assembling sensor devices from a plurality of differently treated gate electrodes, in a similar manner to the high speed ‘pick and place’ of different surface mount electronic components.

The foregoing and other objectives are achieved by the features of the independent claims.

Further implementation forms are apparent from the dependent claims, the description and the Figures.

A first aspect of the present disclosure provides a structure comprising multiple individually removable functionalised electrodes, each one of the multiple individually removable functionalised electrodes for use in at least one biosensor apparatus.

Accordingly, immobilisation of the bio-sensing materials can be carried out in bulk and tested prior to assembly on a substrate whose processing does not have to consider the delicate nature of the bio-sensing materials. By these means, sensors using many tens or hundreds of different sensing materials can be effectively implemented.

In an implementation of the first aspect, respective ones of the multiple removable functionalised electrodes may comprise gate electrodes, each gate electrode comprising at least one surface defining a biosensing layer.

The biosensing layer may comprise an immobilised layer of a selected protein, DNA, or any other recognition material.

The selected protein may be different for each of the multiple individually removable functionalised gate electrodes.

Each of the multiple individually removable functionalised electrodes may be provided in a hermetically sealed structure.

Each of the multiple individually removable functionalised electrodes may be a gate electrode for an extended-gate field-effect transistor, EGFET, or an electrolyte-gated organic field-effect transistor, EGOFET, electrochemical OFET, or electrode of an impedance measurement cell.

Each of the multiple individually removable functionalised electrodes may comprise a mounting portion to mount to or on a biosensor apparatus.

A second aspect of the present disclosure provides a method for manufacturing multiple individually removable functionalised electrodes for use in at least one biosensor apparatus, the method comprising fabricating a set of electrode structures on a substrate, exposing the electrode structures to a biological sensing material, and sealing the so exposed electrode structures in a hermetically sealed structure.

In an implementation of the second aspect, the method may further comprise exposing the electrode structures to a binding material configured to immobilise the biological sensing material on a surface thereof.

Each of the electrode structures may be individually removable from the substrate.

Each of the electrode structures may be a gate electrode for an extended-gate field-effect transistor, EGFET, or an electrolyte-gated organic field-effect transistor, EGOFET.

The method may further comprise processing the substrate to separate individual gate electrode structures.

The method may further comprise processing the substrate to define frangible regions between individual gate electrode structures.

A third aspect of the present disclosure provides a hermetically sealed receptacle comprising multiple individually removable functionalised electrodes for use in at least one biosensor apparatus.

In an implementation of the third aspect, the receptacle may define a cavity sealed with a removable lid.

A fourth aspect of the present disclosure provides a biosensor apparatus comprising multiple sensing sites, each sensing site defining a region configured to receive a functionalised electrode selected from a structure as described herein.

In an implementation of the fourth aspect, each sensing site may comprise a via forming an opening extending from one face of a substrate of the biosensor apparatus to an opposing face of the substrate of the biosensor apparatus.

At least one of the one face of the substrate and the opposing face of the substrate may comprise a conductive trace configured to electrically connect to a functionalised electrode received at a sensing site.

A functionalised electrode for a sensing site may be selected on the basis of an analyte to be sensed.

These and other aspects of the invention will be apparent from the embodiment(s) described below.

### BRIEF DESCRIPTION OF THE DRAWINGS

In order that the present invention may be more readily understood, embodiments of the invention will now be described, by way of example, with reference to the accompanying drawings, in which:

FIG. 1 is an illustration of a typical odourant binding protein, for understanding of the invention;

FIG. 2 is a schematical representation of an EGOFET for understanding of the invention;

FIG. 3 is a schematic representation of gold pins being inserted into a temporary structure for external processing according to an example;

FIG. 4 is a depiction of typical immobilisation of proteins or enzymes on the electrode surface according to an example;

FIG. 5 is a schematic representation showing assembly of a sensing device according to an example;

FIG. 6 is a depiction of wafer scale processing of gate electrodes according to an example;

FIG. 7 is a depiction of tape processing according to an example;

FIG. 8 is a schematic representation of a structure according to an example; and

FIG. 9 is a flow chart of a method for manufacturing multiple individually removable functionalised electrodes for use in at least one biosensor apparatus according to an example.

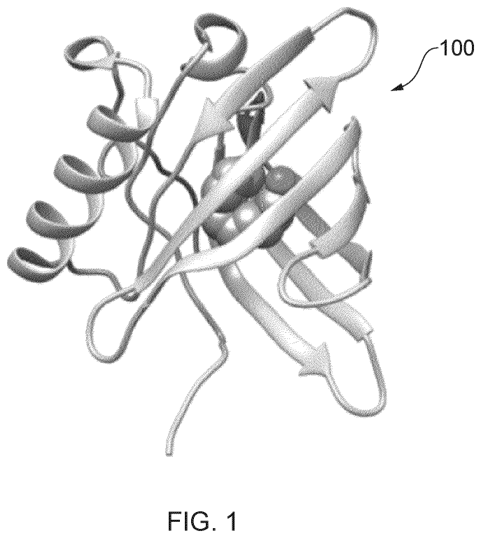

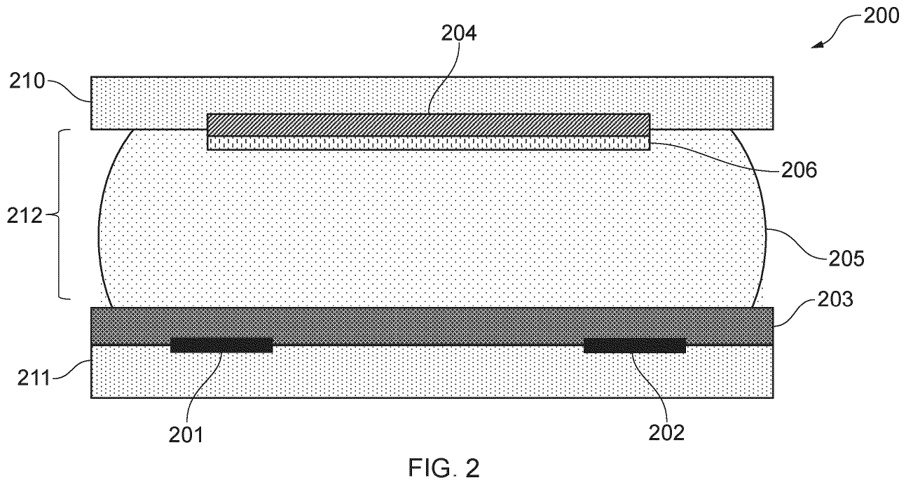

### DETAILED DESCRIPTION

Example embodiments are described below in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

The present disclosure is described with reference to flow charts and/or block diagrams of the method, devices and systems according to examples of the present disclosure. Although the flow diagrams described above show a specific order of execution, the order of execution may differ from that which is depicted. Blocks described in relation to one flow chart may be combined with those of another flow chart. In some examples, some blocks of the flow diagrams may not be necessary and/or additional blocks may be added. It shall be understood that each flow and/or block in the flow charts and/or block diagrams, as well as combinations of the flows and/or diagrams in the flow charts and/or block diagrams can be realized by machine readable instructions.

Current techniques to apply multiple bio sensing proteins (Odorant Binding Proteins, OBP) to the EGOFET devices used in the narcotic detection system are applied directly to the gate electrodes fabricated on a substrate. If the gate electrode is permanently attached to the substrate before immobilisation of the sensing proteins, antibodies or other material, then the deposition, particularly from a fluid, of multiple materials on the same substrate in a small space becomes problematic and costly. In this invention, the immobilisation of the biosensing material can be separated from the final substrate by processing a small gate electrode ‘component’ in bulk, before assembling it into the final substrate.

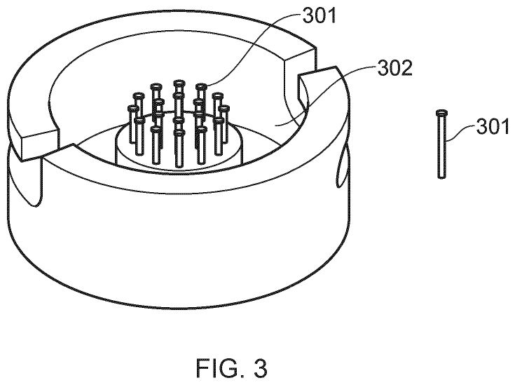

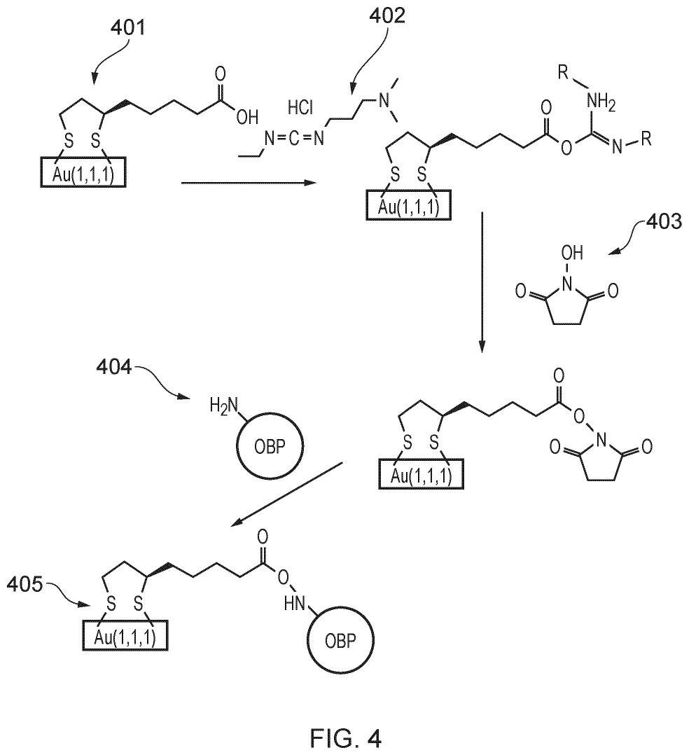

FIG. 3 is a schematic representation of gold pins being inserted into a temporary structure for external processing according to an example. FIG. 4 is a depiction of typical immobilisation of proteins, DNA, RNA, or enzymes etc., on the electrode surface according to an example. According to an example, a functionalised electrode may comprise a coated gold metal pin. Multiple gold coated metal pins (MillMax 6547-0-00-21-00-00-33-0) 301 may be first temporarily mounted in a structure 302. This structure may then be cleaned using 200W Oxygen plasma (Tegal 415). followed by rinsing in deionised water and ethanol. A solution of 1,2-dithiolane-3-pentanoic (lipoic) acid may be applied by immersing the structure in a fluid chamber to form a self-assembled monolayer (SAM) of acid groups on the gold surface 401 over a period of 6-24 hours. After rinsing with ethanol/deionised water, the gold surface may be further modified by immersion in 10 nM of EDC (1-Ethyl-3-[3-dimethylaminopropyl]carbodiimide hydrochloride) 402 and NHS (N-Hydroxysulfosuccinimide) 403 to form an active surface. After further rinsing, an odourant binding protein 404 may be introduced in a buffered phosphate solution, and immobilised to the gold surface 405 by amine binding to the EDC/NHS, followed by rinsing with deionised water and drying. Thus. all pins can be efficiently and reliably coated with the biosensing protein. Multiple structures containing many pins can be coated with different proteins and reference materials. Once coated, these can be stored in hermetically sealed/frozen containers until required, either in the processing structure, or transferred to a storage one.

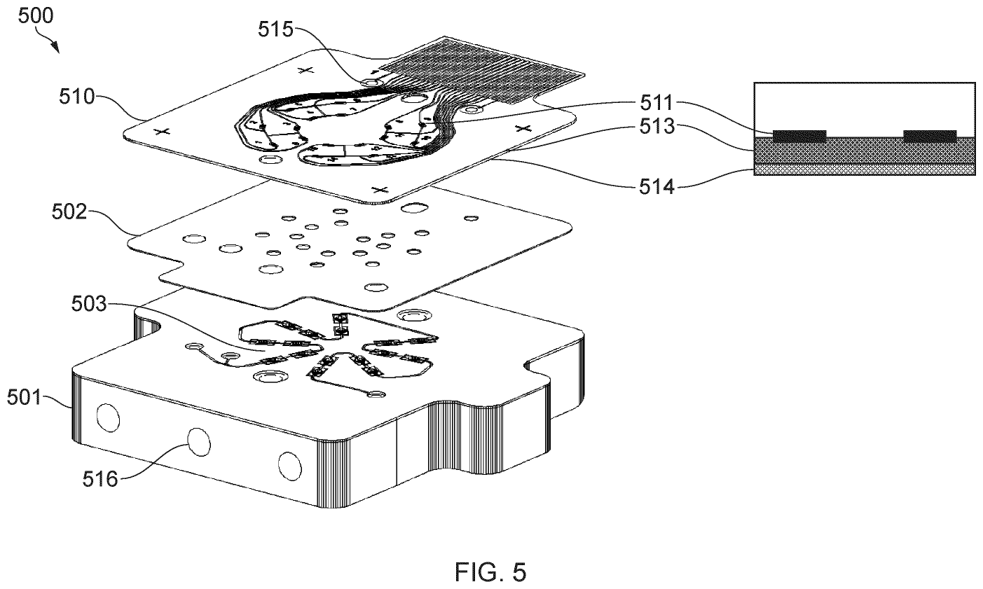

FIG. 5 is a schematic representation showing assembly of a sensing device 500 according to an example. A first substrate 501 with microfluidic channels may be covered with an assembly adhesive 502. Pins may be removed from a plurality of temporary structures 302 and inserted into holes 503 into the substrate. A second substrate 510 may be prepared by patterning electrodes 511 into interdigitated source/drains and interconnect, with a suitable dielectric layer and a coating of a suitable organic semiconductor 513, such as P3HT (poly(3-hexylthiophene-2,5-diyl), to form FET devices. Optionally, a thin dielectric 514, such as Cytop (Asahi Glass), can be used to provide a barrier layer to prevent degradation of the semiconductor. The second substrate 510 may be aligned and laminated to the first substrate 501 to complete the device 500. Connections to the pins forming the gate electrode of the EGOFET devices can either be made directly through the back of the first substrate 501, or conductive material can be deposited, with a via 515 used to connect to a common interconnect on the second substrate 510.

Analyte fluid 516 may be introduced via fluidic ports into the microfluidic channels, and the individual devices transfer characteristics may be determined. When the target analytes are introduced, the current response of the devices with suitable OBPs will change and the matrix of the responses of different proteins can be analysed to provide discrimination and concentration outputs.

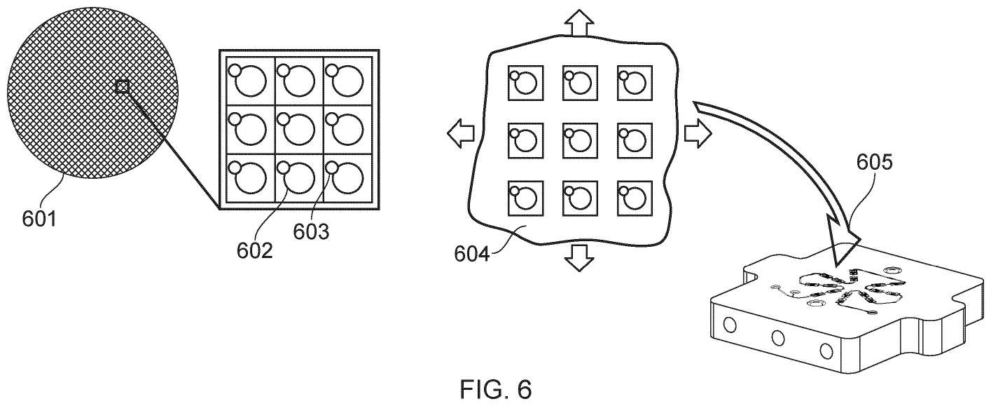

FIG. 6 is a depiction of wafer scale processing of gate electrodes according to an example. A thin glass or silicon wafer structure 601 may be coated with gold, or another suitable metal, and patterned to form a large number of discrete electrodes 602. Vias 603 may be laser or plasma drilled in each electrode and electroplated to form a contact to the opposite side of the wafer. The wafer may be mounted on a flexible film and diced and stretched into individual dies 604. Proteins or other bio-sensing materials may then be immobilised onto the electrode surface in a similar bulk manner to the example of FIGS. 3 and 4 on all the dies and stored. Any suitable recognition material may be used, i.e. any material which is marking, tagging or generally interacting in an observable manner with the analyte of interest. During sensor device fabrication, electrode/dies may be plucked from the film and inserted into the substrate 605 ready for assembly to the top substrate.

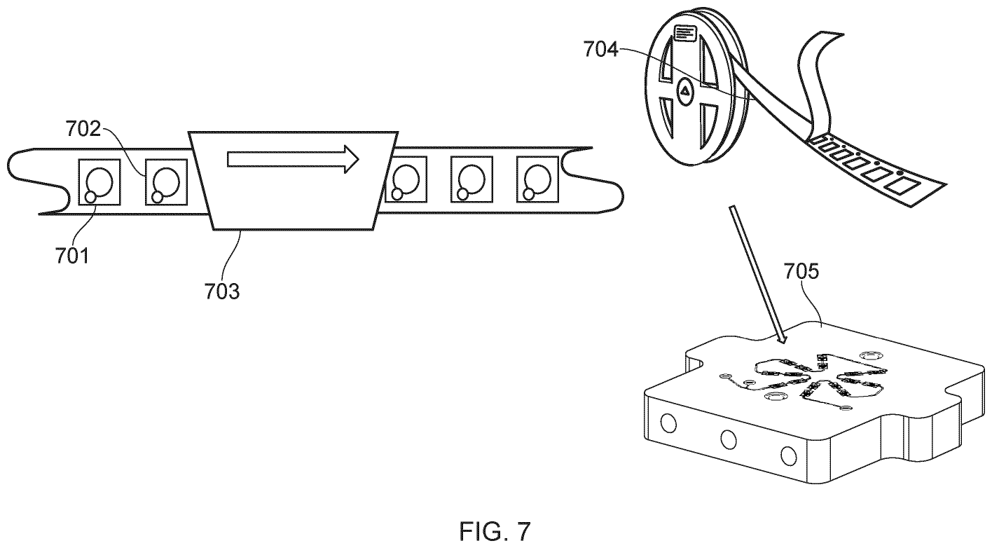

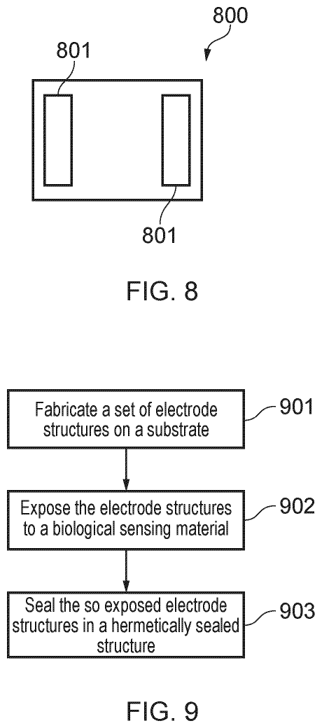

FIG. 7 is a depiction of tape processing according to an example. A continuous tape structure 701 may be processed in a reel-to-reel fashion with metal deposited and patterned using normal lithography 702. Processing of the immobilisation of the proteins or other biosensing materials can be carried out in a semi-continuous fashion with immersion in process chemicals and cleaning 703, with re-reeling or spooling between processes 704. During assembly the coated electrodes may be cut from the tape 701 and placed onto the device substrate using conductive adhesive 705. FIG. 8 is a schematic representation of a structure according to an example. The structure 800 comprises multiple individually removable functionalised electrodes 801, each one of the multiple individually removable functionalised electrodes 801 for use in at least one biosensor apparatus. A “functionalised” electrode comprises an electrode which has been exposed to a specific factor such that it will react a certain way when it subsequently comes into contact with something else. For example, an electrode (such as an electrode comprising a pin) can be exposed to, for example, a protein to functionalise it (i.e., to render it suitable for a selected function, such as detection of something that will bind with the protein). In an example, the protein may effectively stick to the electrode. This effect of this can be amplified by the use of a binding material (i.e., binding material sticks to electrode, and protein sticks to binding material). While FIG. 8 shows two electrodes 801, the skilled person would appreciate that the invention is not limited thereto. The electrodes 801 are considered to be “removable” in the sense that they can be individually removed from the structure 800. Accordingly, the invention enables the electrodes 801 to be prepared as a separate component and inserted into a microfluidic device shortly before assembly, in a similar way to a surface mounted or inserted pin component in an electronic assembly. This has the advantage that many different materials can be prepared in bulk and then assembled. In particular, multiple individually removable electrodes 801 can be fabricated in a container and functionalised such that they react to a specific biological marker (e.g. a drug), and individually selected and removed to then be applied to a device.

Respective ones of the multiple removable functionalised electrodes 801 may comprise gate electrodes, and each gate electrode may comprise at least one surface defining a biosensing layer. The biosensing layer may comprise an immobilised layer of a selected protein. The selected protein may be different for each of the multiple individually removable functionalised gate electrodes 801. Typically, the immobilisation of a biosensing material such as an ‘odour binding protein’ (OBP) may be carried out by initially coating the metal (gold) surface of the gate electrode with a monolayer of a small molecule (such as lipoic acid, DTSSP etc), and optionally activating that surface with a binding agent such as EDC-NHS. This results in a surface which can readily couple to any protein which is further deposited using an aqueous or solvent based dilution, to facilitate a close packing of a single layer of proteins onto the surface.

Each of the multiple individually removable functionalised electrodes 801 may be provided in a hermetically sealed structure. Each of the multiple individually removable functionalised electrodes 801 may be a gate electrode for an extended-gate field-effect transistor, EGFET, or an electrolyte-gated organic field-effect transistor, EGOFET. Each of the multiple individually removable functionalised electrodes 801 may comprise a mounting portion to mount to or on a biosensor apparatus.

FIG. 9 is a flow chart of a method for manufacturing multiple individually removable functionalised electrodes for use in at least one biosensor apparatus according to an example. In block 901, the method comprises fabricating a set of electrode structures on a substrate. In block 902, the method comprises exposing the electrode structures to a biological sensing material. In block 903, the method comprises sealing the so exposed electrode structures in a hermetically sealed structure. The method may further comprise exposing the electrode structures to a binding material configured to immobilise the biological sensing material on a surface thereof. Each of the electrode structures may be individually removable from the substrate. Each of the electrode structures may be a gate electrode for an EGFET or an EGOFET. The method may further comprise processing the substrate to separate individual gate electrode structures. The method may further comprise processing the substrate to define frangible regions between individual gate electrode structures.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the instant disclosure. The embodiments disclosed herein should be considered in all respects illustrative and not restrictive. Reference should be made to the appended claims and their equivalents in determining the scope of the instant disclosure.

1. A structure comprising multiple individually removable functionalised electrodes, each one of the multiple individually removable functionalised electrodes for use in at least one biosensor apparatus.

2. The structure as claimed in claim 1, wherein respective ones of the multiple removable functionalised electrodes comprise gate electrodes, each gate electrode comprising at least one surface defining a biosensing layer.

3. The structure as claimed in claim 2, wherein a biosensing layer comprises an immobilised layer of a selected protein, DNA, or a recognition material.

4. The structure as claimed in claim 3, wherein the selected protein is different for each of the multiple individually removable functionalised gate electrodes.

5. The structure as claimed in claim 1, wherein each of the multiple individually removable functionalised electrodes is provided in a hermetically sealed structure.

6. The structure as claimed in claim 1, wherein each of the multiple individually removable functionalised electrodes is a gate electrode for an extended-gate field-effect transistor, EGFET, an electrolyte-gated organic field-effect transistor, EGOFET, an electrochemical OFET, or an electrode of an impedance measurement cell.

7. The structure as claimed in claim 1, wherein each of the multiple individually removable functionalised electrodes comprises a mounting portion to mount to or on a biosensor apparatus.

8. A method for manufacturing multiple individually removable functionalised electrodes for use in at least one biosensor apparatus, the method comprising:
fabricating a set of electrode structures on a substrate;
exposing the electrode structures to a biological sensing material; and
sealing the so exposed electrode structures in a hermetically sealed structure.

9. The method as claimed in claim 8, further comprising:
exposing the electrode structures to a binding material configured to immobilise the biological sensing material on a surface thereof.

10. The method as claimed in claim 8, wherein each of the electrode structures are individually removable from the substrate.

11. The method as claimed in claim 8, wherein each of the electrode structures is a gate electrode for an extended-gate field-effect transistor, EGFET, or an electrolyte-gated organic field-effect transistor, EGOFET.

12. The method as claimed in claim 8, further comprising:
processing the substrate to separate individual gate electrode structures.

13. The method as claimed in claim 8, further comprising:
processing the substrate to define frangible regions between individual gate electrode structures.

14. Apparatus comprising a hermetically sealed receptacle comprising multiple individually removable functionalised electrodes for use in at least one biosensor apparatus.

15. The apparatus as claimed in claim 14, wherein the receptacle defines a cavity sealed with a removable lid.

16. A biosensor apparatus comprising multiple sensing sites, each sensing site defining a region configured to receive a functionalised electrode selected from a structure as claimed in claim 1.

17. The biosensor apparatus as claimed in claim 16, wherein each sensing site comprises a via forming an opening extending from one face of a substrate of the biosensor apparatus to an opposing face of the substrate of the biosensor apparatus.

18. The biosensor apparatus as claimed in claim 17, wherein at least one of the one face of the substrate and the opposing face of the substrate comprises a conductive trace configured to electrically connect to a functionalised electrode received at a sensing site.

19. The biosensor apparatus as claimed in claim 16, wherein a functionalised electrode for a sensing site is selected on the basis of an analyte to be sensed.
